(19) United States
(12) Patent Application Publication
(10) Pub. No.: US 2024/0315986 A1
(43) Pub. Date: Sep. 26, 2024

(54) PHARMACEUTICAL COMPOSITION FOR TREATMENT OF MULTIPLE SYSTEM ATROPHY

(71) Applicant: THE UNIVERSITY OF TOKYO, Tokyo (JP)

(72) Inventors: Shoji TSUJI, Tokyo (JP); Jun MITSUI, Tokyo (JP)

(21) Appl. No.: 18/588,446

(22) Filed: Feb. 27, 2024

Related U.S. Application Data
(63) Continuation-in-part of application No. PCT/JP2022/032696, filed on Aug. 31, 2022.

(30) Foreign Application Priority Data
Aug. 31, 2021 (JP) 2021-140886

(51) Int. Cl. A61K 31/09; A61K 9/00

(52) U.S. Cl. CPC A61K 31/09; A61K 9/0053

(57) ABSTRACT

Provided is a pharmaceutical composition that can treat multiple system atrophy by alleviating or suppressing the progression of the various symptoms caused by multiple system atrophy. Provided is a pharmaceutical composition for treatment of multiple system atrophy, the composition containing ubiquinol, in which the ubiquinol is subjected to oral administration repeatedly for 7 days or more at a dose of 1,500 to 1,800 mg per day.

### BACKGROUND OF THE INVENTION

### 1. Technical Field

The present invention relates to a pharmaceutical composition for treatment of multiple system atrophy by a repeated oral administration of ubiquinol at a high dose.

### 2. Description of the Related Art

The multiple system atrophy (MSA) is one of progressive and intractable neurodegenerative diseases with poor prognosis. A mean age of onset of the MSA is 57.5±7.2 years old, with clinical syndromes of cerebellar ataxia, parkinsonism, autonomic failure, and pyramidal signs appearing in various combinations. Among the clinical presentations, there are two representative clinical disease types: MSA-C with the cerebellar ataxia as a major clinical presentation and MSA-P with the parkinsonism as a major clinical presentation. The MSA-C corresponds to olivopontocerebellar atrophy and the MSA-P corresponds to striatonigral degeneration, which are traditional nomenclatures. In addition, there is Shy-Drager syndrome with autonomic dysfunction as a major syndrome. In the Japanese population, a frequency of the MSA-C is higher than that of the MSA-P, and the MSA-C occurs at a frequency of 8,000 and the MSA-P occurs at a frequency of 4,000 with respect to total number of MSA patients of 12,000. In contrast, the frequency of the MSA-P is higher in the European-descent population. Actually, there may be patients with the symptom of the MSA-P overlapped with the symptom of the MSA-C and exhibiting both symptoms, in which case the patients cannot be clearly classified.

Coenzyme Q₁₀ (CoQ₁₀) includes an oxidized form (ubiquinone) and a reduced form (ubiquinol), and the ubiquinol exhibits a pharmacological action in vivo (Raizner, et al. Coenzyme Q10. Methodist Debakey Cardiovasc J. 2019; 15(3):185-191). The ubiquinol is an unstable substance that is easily oxidized. Therefore, application of the ubiquinone to various neurological disease treatments has been attempted so far. The ubiquinone is converted to the ubiquinol by reductases such as a NADPH-dependent CoQ reductase, a DT diaphorase, a lipoamide dehydrogenase, and a thioredoxin reductase present in cytoplasm (Turunen, et al. Metabolism and function of coenzyme Q. Biochim Biophys Acta. 2004 28; 1660(1-2):171-199)). Accordingly, even in a case where the ubiquinone (oxidized) is subjected to intravenous administration to a rat and oral administration to a human, the ubiquinoe is converted to the ubiquinol, hence the ubiquinol (reduced) of 95% or more is present in plasma (Watanabe, et al. PET imaging of 11C-labeled coenzyme Q10: Comparison of biodistribution between [11C]ubiquinol-10 and [11C]ubiquinon-10. Biochem Biophys Res Commun. 2019 May 7; 512(3):611-615 and Langsjoen, et al. Comparison study of plasma coenzyme Qi0 levels in healthy subjects supplemented with ubiquinol versus ubiquinone. Clin Pharmacol Drug Dev. 2014; 3(1):13-17).

What has been reported includes, confirmation of an amount of CoQ10 decreasing in tissues of patients with familial and sporadic MSA, strong suggestion of logical therapeutic effects on the MSA by administration of the CoQ10, and an example that a clinical trial has been conducted by repeatedly administering the ubiquinol to a patient with familial MSA carrying R337X/V343A (nomenclature based on NM_001358921.2) or R387X/V393A (nomenclature based on NM_015697.9) compound heterozygous pathogenic variants in the COQ2 gene (JP 6358962 B2).

The same literature describes a clinical evaluation scale (FIG. 9) when 600 mg for 2 weeks, subsequently 840 mg for 2 weeks, and further 1,200 mg for 2 weeks of the ubiquinol were repeatedly administered once per day from a gastrostomy tube, but describes in paragraph 0085 that “there were minor variations, but no statistically significant changes in the clinical evaluation scale were confirmed.”

### SUMMARY OF THE INVENTION

Causes of the development of the MSA are unknown, and there has been no effective therapy for suppressing progression of the symptoms so far. In a clinical trial described in JP 6358962 B2 for one familial MSA patient having an extremely rare compound heterozygous pathogenic variants in the COQ2 gene, it can be said that a sign of non-zero but extremely minute therapeutic effects on the multiple system atrophy including improved brain oxygen consumption rate reflecting improved mitochondrial function has been confirmed by administering the ubiquinol in an amount exceeding a conventional dose for a certain period or longer.

This is a valuable result considering that there has been no therapy so far, but it is not satisfactory from a viewpoint of the result obtained from one patient at an advanced clinical stage. Further, since the trial is for the familial MSA patient having the extremely rare compound heterozygous pathogenic variants but not for general sporadic multiple system atrophy patients, therapeutic effects for the sporadic multiple system atrophy patients are unknown, and it is desirable to develop effective therapies and therapeutic drugs.

Under such background, the present inventors have made intensive studies, and have found that a concentration of the ubiquinol in the plasma can be substantially increased by administering a conventionally unforeseeable amount of the ubiquinol for a conventionally unforeseeable long period of time, and as a result, the symptoms of the multiple system atrophy can be alleviated at an unexperienced level so far to arrive at the present invention.

The present invention has been made in view of such circumstances, and an object of the present invention is to provide a pharmaceutical composition with which various symptoms due to the multiple system atrophy can be relieved or progression of the symptoms can be suppressed, and the multiple system atrophy can be treated by suppressing the progression of the multiple system atrophy.

In other words, the present invention is to provide embodiments below.

Embodiment 1: A pharmaceutical composition for treatment of multiple system atrophy, the composition containing ubiquinol, in which the ubiquinol is subjected to oral administration repeatedly for 7 days or more at a dose of 1,500 to 1,800 mg per day.

Embodiment 2: The pharmaceutical composition according to Embodiment 1, in which the dose of the ubiquinol per day is increased or reduced in a range of 1,500 to 1,800 mg.

Embodiment 3: The pharmaceutical composition according to Embodiment 1, in which the dose of the ubiquinol per day is 1,500 mg.

Embodiment 4: The pharmaceutical composition according to any one of Embodiments 1 to 3, in which the composition is subjected to the oral administration repeatedly for 14 days or more.

Embodiment 5: The pharmaceutical composition according to any one of Embodiments 1 to 4, in which a maximum plasma concentration of the ubiquinol is 10 μg/mL or more after Day 7 from start of the administration.

Embodiment 6: The pharmaceutical composition according to Embodiment 5, in which the maximum plasma concentration of the ubiquinol is about 15 μg/mL after Day 7 from the start of the administration.

Embodiment 7: The pharmaceutical composition according to any one of Embodiments 1 to 6, in which the multiple system atrophy is type C (MSA-C).

Embodiment 8: The pharmaceutical composition according to any one of Embodiments 1 to 7, in which the composition suppresses or alleviates one or more symptoms due to the multiple system atrophy selected from the group of parkinsonism, cerebellar ataxia, autonomic failure, and pyramidal signs.

Embodiment 9: A therapeutic agent for multiple system atrophy by setting a maximum plasma concentration of ubiquinol to 10 μg/mL or more.

Embodiment 10: The therapeutic agent according to Embodiment 9, in which the maximum plasma concentration of the ubiquinol is about 15 μg/mL.

According to the present invention, it is possible to provide the pharmaceutical composition with which the multiple system atrophy can be treated by relieving or suppressing the progression of the various symptoms of the multiple system atrophy whose progression of the symptoms cannot be suppressed by an effective therapy so far.

### BRIEF DESCRIPTION OF THE DRAWINGS

FIG. 1 is a graph showing measurement results of concentrations of a drug in plasma in a placebo group and an MSA-01 group (900 mg, 1,200 mg, and 1,500 mg) during a repeated oral administration for 14 days in Test Example 1;

FIG. 2 is a diagram showing drug ascending administration protocols of the placebo group and the MSA-01 group in Test Example 2;

FIGS. 3A and 3B are graphs showing changes of UMSARS part 2 scores (FIG. 3A) and Barthel indices (FIG. 3B) from baselines at Weeks 12, 24, 36, and 48 in Test Example 2; and

FIG. 4 is a table summarizing the changes from the baselines at Week 48 for each evaluation item in Test Example 2.

### DETAILED DESCRIPTION

Hereinafter, the pharmaceutical composition of the present invention will be specifically described.

The pharmaceutical composition of the present invention is a pharmaceutical composition for treatment of multiple system atrophy by a repeated oral administration of ubiquinol at a high dose.

### Ubiquinol

The ubiquinol is 2-[(2E,6E,10E,14E,18E,22E,26E,30E,34E)-3,7,11,15,19,23,27,31,35,39-decamethyltetraconta-2,6,10,14,18,22,26,30,34,38-decaenyl]-5,6-dimethoxy-3-methylcyclohexa-2,5-diene-1,4-diol, and is a reduced coenzyme Q₁₀(CoQ₁₀). In the CoQ₁₀, the ubiquinol exhibits a pharmacological action (mainly, an antioxidant action and enhancement of mitochondrial function. As the ubiquinol, it is possible to use, for example, one that is obtained by a fermentation method, a chemical synthesis method, or an extraction method from animals and plants, but one having an all-trans structure to be obtained by the fermentation method is preferable from a viewpoint of a biological use, and for example, Kaneka Coenzyme Q₁₀ (manufactured by Kaneka Corporation) can be used.

### Multiple System Atrophy (MSA)

Specific symptoms include symptoms due to cerebellar disorders such as gait disturbance and difficulty in talking, Parkinsonian syndrome such as tremor, rigidity, and difficulty in movement, and symptoms caused by autonomic nervous system such as fluctuations in a blood pressure or dysfunctions in urination and defecation; and cause disorders in many nervous systems. At present, it is estimated that there are about 10 patients per 100,000 people (the number of the patients of about 12,000) in Japan. Since an onset age of the disease is typically in late 50s on average, in the prime of life, and the disease progresses relatively quickly, the disease is one of neurological diseases that cause a large social loss (2013/6/12 Finding important genetic factors of intractable neurological diseases and multiple system atrophy due to unknown causes and paving a way for implementation of therapies by The University of Tokyo Hospital—implementation for the first time by large-scale international multi-center cooperative research —). Accordingly, delaying the progression of the disease, for example, about 25%, is an effective therapy and improves QOL of the patients.

### Prognosis of Multiple System Atrophy (MSA)

Since striatum is degenerated in the multiple system atrophy, effects of antiparkinson drugs are hardly effective. Further, since cerebellar symptoms and the autonomic neuropathy are also included, the symptoms are usually exacerbated in a progressive manner as a whole. According to results of a study on 230 patients in Japan, it has been reported that after the onset as a median, the patients use wheelchairs within about 5 years, and entered a bedridden state within about 8 years, and duration of the disease is about 9 years (Japan Intractable Disease Information Center: Multiple System Atrophy (1) Striatonigral Degeneration, a subtype of MSA, (Designated Intractable Disease 17), https://www.nanbyou.or.jp/entry/221).

### Treatment of Multiple System Atrophy (MSA)

Currently, there is no effective therapy for the MSA, but a symptomatic therapy is taken for each symptom as follows. For the ataxia, prothyrelin (Hiltonin (registered trademark)) which is a thyrotropin releasing hormone derivative (0.5 to 2 mg, intramuscular or intravenous injection, 2 to 3 weeks) or taltirelin (Ceredist (registered trademark)) (5 mg, oral, twice per day) is administered [1]. For the parkinsonism, it can be said that, the effects of the antiparkinson drugs are hardly obtained with only some patients showing response to levodopa and in many cases, the treatment responsiveness disappears within a few years. For orthostatic hypotension, salt and water are replenished. An intravascular volume can be sometimes increased by administration of fludrocortisone (0.1 to 0.4 mg, oral, once per day) [2]. Further, wearing of a tourniquet on a lower half of a body and a adrenoceptor stimulation by midodrine (10 mg, oral, 3 times per day) can be useful [2]. However, the midodrine increases peripheral vascular resistance and raises a decubitus blood pressure, which may raise a problem. Raising a head side of a bed by about 10 cm may reduce nocturia and decubitus hypertension, and reduce morning orthostatic hypotension. For constipation, a high-fiber diet and a fecal softening agent can be used, but an enema may be required in intractable cases [3]. For urinary incontinence, oxybutynin chloride (5 mg, oral, 3 times per day) or tolterodine (2 mg, oral, twice per day) may be used as long as a cause of the urinary incontinence is hyperreflexia of a detrusor muscle [4]. For urinary urge, administration of tamsulosin (0.4 to 0.8 mg, once per day) may be effective [5]. For urinary retention, self-catheterization with a catheter is required in many patients [5]. For erectile dysfunction, pharmacotherapy such as sildenafil (50 mg, oral, single dose) or tadalafil (2.5 to 5 mg, once per day) and various physical approaches can be used [5].

### Pharmaceutical Composition

The pharmaceutical composition of the present invention is one that is subjected to the oral administration repeatedly with the ubiquinol at a dose of 1,500 to 1,800 mg per day for 7 days or more. The dose of the ubiquinol per day may ascend, or increase or decrease between 1,500 to 1,800 mg during the administration, or the ubiquinol may be continuously administered at a constant dose. Examples of a preferred dose include 1,500 mg per day.

It is preferable that an administration period of the pharmaceutical composition of the present invention is 7 days or more, and continuous administration is performed once to three times per day until a point of time when the maximum plasma concentration of the ubiquinol is for example about 4 μg/mL or more, about 6 μg/mL or more, about 8 μg/mL or more, about 10 g/mL or more, about 12 μg/mL or more, or about 15 μg/mL or more. Since the therapeutic effects are exerted by maintaining the plasma concentration of the ubiquinol to be for example about 4 μg/mL or more, about 6 μg/mL or more, about 8 μg/mL or more, about 10 μg/mL or more, about 12 μg/mL or more, or about 15 μg/mL or more, it is preferable to continue the administration while maintaining such plasma concentration. Specifically, the administration period can be, for example, 7 days, 14 days, 28 days, 35 days, 42 days, 56 days, 84 days, 168 days, 336 days, or more. In general, since it is unlikely that the MSA will be completely cured, it is preferable to continue taking the composition after the plasma concentration of the ubiquinol reaches 10 μg/mL or more or about 15 μg/mL.

It is known that there are the MSA-C, the MSA-P, and a mixed type thereof in MSA, and there also are a familial disease state due to a specific genotype, and a sporadic disease state that occurs regardless of the genotype. The pharmaceutical composition of the present invention exerts the effects regardless of the type of the MSA, since it is considered that the composition can relieve the symptoms of the MSA and suppress the progression of the MSA by achieving a high maximum plasma concentration by repeatedly administering a high dose of the ubiquinol.

Moreover, the pharmaceutical composition of the present invention can relieve the specific symptoms caused by the MSA, for example, the symptoms due to the cerebellar disorders such as the wobble and the difficulty in the talking, the Parkinsonian syndrome such as tremor, rigidity, and the difficulty in the movement, and the symptoms caused by autonomic nervous system such as the fluctuations in the blood pressure or the dysfunctions in the urination and the defecation; and suppress the progression of the symptoms.

Further, the pharmaceutical composition of the present invention can relieve the various symptoms caused by the MSA and suppress the progression of the symptoms when being administered to patients corresponding to any one of (1) to (3) below.
(1) Patients who are determined with Probable or Possible MSA in accordance with diagnostic criteria from Gilman et al.
(2) Patients who can walk 10 meters or more by himself/herself without an instrument or by himself/herself with an auxiliary tool such as a cane, a pedestrian, or a handrail.
(3) Patients who undergo mutational analysis of the COQ2 gene

The (1) described above is a diagnostic index of the MSA defined by Gilman et al. as of the filing date, and the (2) and the (3) are inclusion criteria used in the Phase 2 study. These diagnostic criteria can be changed in accordance with study progresses of the MSA, but it is considered that the pharmaceutical composition of the present invention is effective in relieving the various symptoms and suppressing the progression of the symptoms by being administered to an MSA patient diagnosed by using a standard diagnostic criterion at a point of time of the administration.

The pharmaceutical composition of the present invention can be in a dosage form suitable for the oral administration, and specific forms are not limited. For example, solid preparations such as powders, granules, tablets, capsules (a hard capsule and a soft capsule), and dry syrups, as well as liquid preparations such as syrups can be used. Such preparations can be produced by a conventionally known method.

### Therapeutic Agent

The present invention relates to a therapeutic agent for the multiple system atrophy by setting the maximum plasma concentration of the ubiquinol to for example about 4 μg/mL or more, about 6 μg/mL or more, about 8 μg/mL or more, about 10 μg/mL or more, about 12 μg/mL or more, or about 15 μg/mL or more. Such maximum plasma concentration can suppress the progression of the multiple system atrophy.

An administration route and a dosage form of the therapeutic agent for the multiple system atrophy are not particularly limited. Examples of the administration route include the oral administration, tubular administration, administration through gastric fistula, and intravenous administration, and examples of the dosage form include the solid preparations such as the powders, the granules, the tablets, the capsules (the hard capsule and the soft capsule), and the dry syrups, the liquid preparations such as the syrups, as well as injections. Such preparations can be produced by a conventionally known method.

### Cited Literature

1. Japanese Society of Neurology, Practical Guidelines for Spinocerebellar Degeneration and Multiple System Atrophy 2018, edited by Committee for Preparation of “Practical Guidelines for Spinocerebellar Degeneration and Multiple System Atrophy,” Nankodo Co., Ltd., Tokyo 2018; p 212-214.
2. Japanese Society of Neurology, Practical Guidelines for Spinocerebellar Degeneration and Multiple System Atrophy 2018, edited by Committee for Preparation of “Practical Guidelines for Spinocerebellar Degeneration and Multiple System Atrophy,” Nankodo Co., Ltd., Tokyo 2018; p 222-224.
3. Japanese Society of Neurology, Practical Guidelines for Spinocerebellar Degeneration and Multiple System Atrophy 2018, edited by Committee for Preparation of “Practical Guidelines for Spinocerebellar Degeneration and Multiple System Atrophy,” Nankodo Co., Ltd., Tokyo 2018; p 228-230.
4. Japanese Society of Neurology, Practical Guidelines for Spinocerebellar Degeneration and Multiple System Atrophy 2018, edited by Committee for Preparation of “Practical Guidelines for Spinocerebellar Degeneration and Multiple System Atrophy,” Nankodo Co., Ltd., Tokyo 2018; p 231-232.
5. Japanese Society of Neurology, Practical Guidelines for Spinocerebellar Degeneration and Multiple System Atrophy 2018, edited by Committee for Preparation of “Practical Guidelines for Spinocerebellar Degeneration and Multiple System Atrophy,” Nankodo Co., Ltd., Tokyo 2018; p 233-234.

### EXAMPLES

Although the present invention is described below with reference to specific embodiments, it is understood that the present invention is not limited to the embodiments, and that various changes and modifications may be made therein by a person skilled in the art without departing from a scope or spirit of the invention as defined in appended claims.

### Test Example 1: Phase 1 Clinical Trial of MSA-01 (Ubiquinol)

### (Measurement of Plasma Concentration of Drug in Repeated Oral Administration for 14 Days)

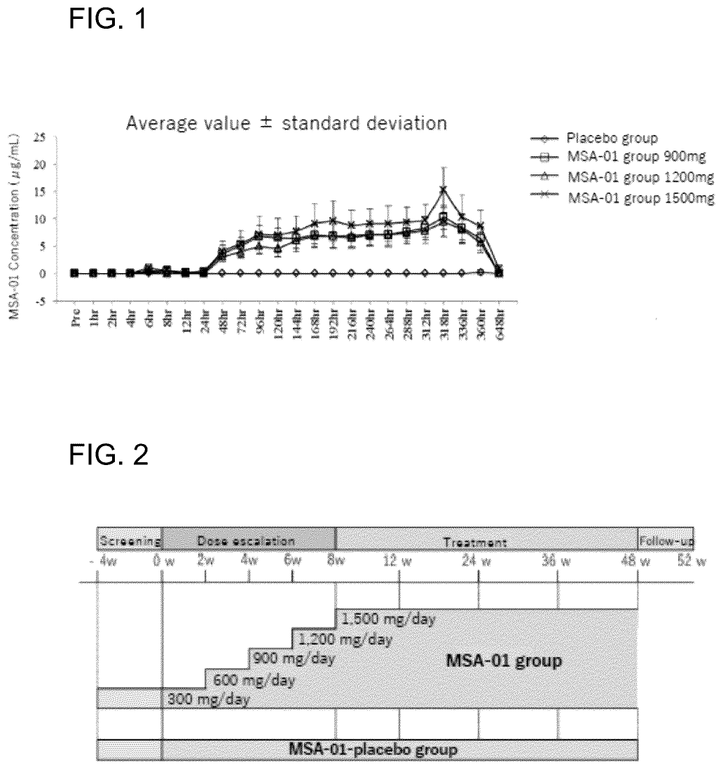

The MSA-01 was subjected to the oral administration to healthy subjects at doses of 900 mg, 1,200 mg, and 1,500 mg repeatedly for 14 days. As a result, the plasma concentration of the MSA-01 until 24 hours after the administration was extremely low (quantification limit: 1 g/mL), but the plasma concentration of the MSA-01 (Level of MSA-01 in plasma) gradually increased by subsequent repeated administration, and reached a steady state by the administration for about 7 days. Maximum plasma concentrations of the MSA-01 detected were 10.24, 9.38, and 15.22 μg/mL in 900 mg, 1,200 mg, and 1,500 mg groups, respectively, in measurement after 6 hours from the repeated oral administration for 14 days (a value at 318 hour in FIG. 1). Published baselines of the plasma concentration of the ubiquinol are 0.87 and 0.66 g/mL (% Reduced CoQ₁₀: 98.9 and 85%) (Langsjoen, et al. Comparison study of plasma coenzyme Q10 levels in healthy subjects supplemented with ubiquinol versus ubiquinone. Clin Pharmacol Drug Dev. 2014; 3(1):13-17, Onur et al. Determination of the coenzyme Q10 status in a large Caucasian study population. Biofactors 2015; 41(4):211-21), and it is estimated that MSA-01 in the plasma increased about 20-fold from the baselines in the 1500 mg group in the test.

### Test Example 2: Phase 2 Clinical Trial of MSA-01 (Ubiquinol)

For a group of 21 patients with COQ2 mutation and a group of 108 patients without COQ2 mutation, 61 patients were administered with the MSA-01 and 68 patients were administered with the placebo for each group. Among 129 patients in total in the patient groups, 99 patients had the MSA-C and 30 patients had the MSA-P. The 129 patients who were confirmed to be eligible during 4-week observation period were randomly assigned to be administered with the MSA-01 or the placebo once per day after breakfast (doses of the MSA-01 or the placebo ascend every 2 weeks to 300, 600, 900, 1,200, and 1,500 mg/day, FIG. 2), and evaluation items according to main evaluation items were measured at Weeks 12, 24, 36, and 48.

### Primary Efficacy Outcome

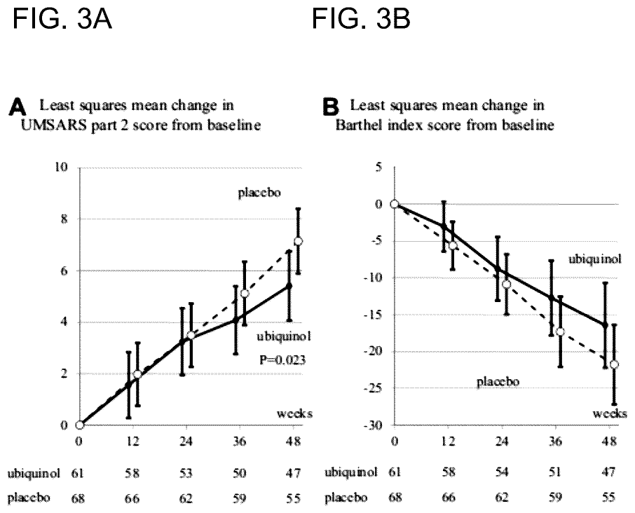

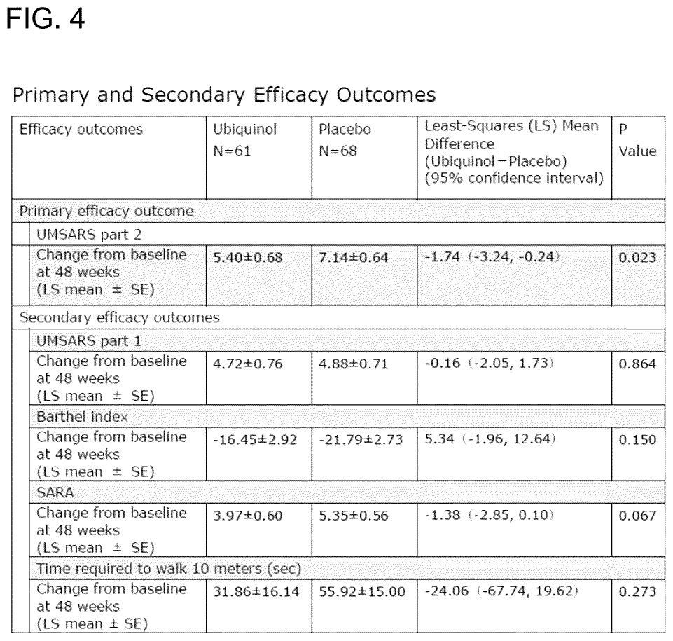

Changes (LS means) of UMSARS part 2 scores at Weeks 12, 24, 36, and 48 from the baseline (unified multiple-system atrophy rating scale part 2; Wenning G K, Tison F, Seppi K, et al. Development and validation of the Unified Multiple System Atrophy Rating Scale (UMSARS). Mov Disord [Internet] 2004; 19(12):1391-402) were evaluated. The LS means are closer to zero indicating more effective, but at week 48, the LS means were 5.40 in the MSA-01 group, 7.14 in the placebo group, and were significantly smaller in the MSA-01 group (with a difference of −1.74; a 95% confidence interval of −3.24 to −0.24; P=0.023). Results are shown in FIG. 3A (at Weeks 12, 24, 36, and 48) and FIG. 4 (at Week 48).

### Secondary Efficacy Outcome

Changes (LS mean) of a UMSARS part 1 score (unified multiple-system atrophy rating scale part 1; Wenning G K, Tison F, Seppi K, et al. Development and validation of the Unified Multiple System Atrophy Rating Scale (UMSARS). Mov Disord [Internet] 2004; 19(12):1391-402), the Barthel index (Barthel index: Mahoney F I, Barthel D W. Functional evaluation: The Barthel index. Md State Med J [Internet] 1965; 14:61-5. Available from:http://www.ncbi.nlm.nih.gov/pubmed/14258950) (FIG. 3B), a SARA score (Schmitz-Huebsch T, du Montcel S T, Baliko L, Berciano J, Boesch S, Depondt C, Giunti P, Globas C, Infante J, Kang J S, Kremer B, Mariotti C, Melegh B, Pandolfo M, Rakowicz M, Ribai P, Rola R, Schoels L, Szymanski S, van de Warrenburg B P, Duerr A, Klockgether T, Fancellu R. Scale for the assessment and rating of ataxia:development of a new clinical scale. Neurology. 2006 Jun. 13; 66(11):1717-20. doi: 10.1212/01.wnl.0000219042.60538.92), and a 10-meter walking time (second) at Week 48 from baselines was evaluated (FIG. 4). As can be seen from FIG. 4, in the evaluation of the Barthel index, the SARA score, and the 10-meter walking time, effects were shown in the MSA-01 group as compared to the placebo group.

According to the present invention, it is possible to provide a pharmaceutical composition and a therapeutic agent with which the various symptoms due to the multiple system atrophy can be treated by relieving the symptoms of the multiple system atrophy and suppressing the progression of the symptoms.

1. A pharmaceutical composition for treatment of multiple system atrophy, the composition comprising ubiquinol, wherein the ubiquinol is subjected to oral administration repeatedly for 7 days or more at a dose of 1,500 to 1,800 mg per day.

2. The pharmaceutical composition according to claim 1, wherein the dose of the ubiquinol per day is increased or reduced in a range of 1,500 to 1,800 mg.

3. The pharmaceutical composition according to claim 1, wherein the dose of the ubiquinol per day is 1,500 mg.

4. The pharmaceutical composition according to claim 1, wherein the composition is subjected to the oral administration repeatedly for 14 days or more.

5. The pharmaceutical composition according to claim 1, wherein a maximum plasma concentration of the ubiquinol is 4 μg/mL or more after Day 7 from start of the administration.

6. The pharmaceutical composition according to claim 5, wherein the maximum plasma concentration of the ubiquinol is about 8 μg/mL or more after Day 7 from the start of the administration.

7. The pharmaceutical composition according to claim 1, wherein the multiple system atrophy is type C (MSA-C).

8. The pharmaceutical composition according to claim 1, wherein the composition suppresses or alleviates one or more symptoms due to the multiple system atrophy selected from the group of parkinsonism, cerebellar ataxia, autonomic failure, and pyramidal signs.

9. A therapeutic agent for multiple system atrophy by setting a maximum plasma concentration of ubiquinol to 4 μg/mL or more.

10. The therapeutic agent according to claim 9, wherein the maximum plasma concentration of the ubiquinol is about 8 μg/mL or more.
